(19) United States
(12) Patent Application Publication
(10) Pub. No.: US 2014/0034705 A1
(43) Pub. Date: Feb. 6, 2014

(54) Tongue debulking stapler and tongue tissue holder

(71) Applicant: Ahmed Mohamed Mohy Eldin El Bassiuony, Kuwait (KW)

(72) Inventor: Ahmed Mohamed Mohy Eldin El Bassiuony, Kuwait (KW)

(21) Appl. No.: 13/507,146

(22) Filed: Sep. 6, 2012

Related U.S. Application Data
(60) Provisional application No. 61/687,068, filed on Apr. 18, 2012.

(51) Int. Cl. A61B 17/068

(52) U.S. Cl. CPC A61B 17/068
USPC 227/181.1; 227/175.1

(57) ABSTRACT

The main idea for the tongue debulking stapler is to introduce a new surgical procedure for tongue size reduction in cases of macroglossia and in concern for cases of moderate and sever OSA as a one of the minimally invasive procedures that will have a better outcome and less intraoperative and postoperative complications than the conventional procedures. The tongue debulking stapler did not used before in the field of otolaryngology. The stapler has 3 parts (hand—body—stapling cutting part) and for each parts there specific protection elements. The stapler is supplied by tongue tissue holder to facilitates its work

### CROSS-REFERENCE TO RELATED APPLICATIONS

“Not applicable”

### STATEMENT REGARDING FEDERALLY SPONSORED RESEARCH OR DEVELOPMENT

“Not applicable”

### SEQUENCE LISTING

“Not applicable”

### THE NAMES OF THE PARTIES TO A JOINT RESEARCH AGREEMENT

“Not applicable”

### BACKGROUND OF THE INVENTION

1. Field of Invention

This invention described herein relates to devices and associated method for treating macroglossia. More specifically, the invention described herein relate to devices and methods for treating enlarged tongue base in the treatment of cases of obstructive sleep apnea as a minimally invasive technique as the previous conventional tongue base surgeries had a lot of intraoperative and postoperative complications and necessities a temporary preoperative tracheostomy

2. Description of the Related Art

Obstructive sleep apnea (osa) is highly prevalent medical problem. One in fifteen adults has moderate to severe osa requiring treatment. Untreated osa results in reduced quality of life and increased risk of disease including hypertension, stroke, heart disease, etc.

Continuous positive airway pressure (cpap) is the standard treatment for osa. while cpap is non-invasive and highly effective, it is not well tolerated by patients. Patient compliance for CPAP is often reported to be between 40% and 60%.

Surgical treatment options for OSA are available too. However, they tend to be highly invasive, irreversible, and have poor and/or inconsistent efficacy. Even the more effective surgical procedures are undesirable because they usually require multiple invasive and irreversible operations, they may alter a patient's appearance (e.g., maxillo-mandibulary advancement), and/or they may be socially stigmatic (e.g., tracheostomy)

The tongue base reduction surgeries, partial glossectomy operations, are one of the curative, but, it is considered as major surgeries for obstructive sleep apnea patients with a lot of perioperative problems

The tongue debulking stapler is a design of a new surgical instrument based on the minimally invasive concept that will be of value in the field of OSA; more specifically; tongue base surgery

The tongue debulking stapler can avoids the drawbacks of partial glossectomy operations mainly the need of preoperative temporary tracheostomy because of the possibility of intraoperative bleeding and postoperative tongue edema

### BRIEF SUMMARY OF THE INVENTION

The present invention provides device and method for tongue base reduction for osa patient asone of the minimally invasive procedures with better outcome and less intraoperative and postoperative complications compared to the other conventional surgical methods as described in the following detailed description.

### BRIEF DESCRIPTION OF THE SEVERAL VIEWS OF THE DRAWING

Together with the following detailed description, the drawings illustrate exemplary embodiments and serve to explain certain principles in the drawings:

FIG. 1: A schematic diagram showing side view and parts of the shape of the tongue debulking stapler; vertical orientation

FIG. 2: A schematic diagram showing another side view and parts of the shape of the tongue debulking stapler; vertical orientation

FIG. 3: A schematic diagram showing two views together; one while the handle is open and the other one while the handle is closed

FIGS. 4 & 5: Schematic diagrams showing parts the handle with view of the stapler cutting segment while the handle is opened.

FIGS. 6 & 7: Schematic diagrams showing the parts handle with view of the stapler cutting segment while the handle closed

FIGS. 8, 9 & 10: Schematic diagrams showing parts of the staper body and movement of the sliding bar in relation to the stapling cutting segment; resting position and while moving forewords when the handle closed

FIGS. 11 & 12: Schematic diagrams showing the stapling cutting segment; opened and closed

FIGS. 13 & 14: Schematic diagrams showing front view of the stapling cutting segment; while the stapler is in vertical position

FIGS. 15 & 16: Schematic diagrams showing front view of the stapling cutting segment; while the stapler is in horizontal position

FIGS. 17 & 18: Schematic diagrams showing the direction of the cut section and the cut section of the stapler while the stapler is in vertical position

FIGS. 19 & 20: Schematic diagrams showing the direction of the cut section and the cut section of the stapler while the stapler is in horizontal position

FIGS. 21, 22 & 23: Schematic diagrams showing projection views of the strut holding the cutter and the pushing arm of the staples

FIG. 24: A schematic diagram showing the details of the staples holding segment

FIG. 25: A schematic diagram showing the staple

FIG. 26: A schematic diagram showing parts of the tongue tissue holder

### BRIEF SUMMARY OF THE INVENTION

The main idea for the tongue debulking stapler is to make surgical procedure for tongue base reduction in cases of moderate and sever OSA one of the minimally invasive procedures with better outcome and less intraoperative and postoperative complications than the conventional procedures. The tongue debulking stapler did not used before in the field of otolaryngology. The stapler has 3 parts (handle—body and stapling cutting part) and for each part there specific protection elements. The stapler is supplied by tongue tissue holder to facilitates its work

### DETAILED DESCRIPTION OF THE INVENTION

Partial glossectomy is a surgical procedure to remove part of the tongue tissue. It can be performed for treatment of different causes of tongue enlargement. Enlargement of the back part of tongue (called the tongue base) can lead to significant obstruction of the upper airway during sleep. Partial glossectomy or tongue base reduction involves removing a portion of the back part of tongue. This results in less tissue bulk to fall backwards to block your breathing. The airway in the back of your throat is opened, allowing for more comfortable sleeping at night.

The tongue debulkinge stapler is a new design of surgical instrument that will be used in the field of tongue base reduction surgeries in cases of moderate to severe and severe cases of OSA

The idea is to be one of the minimally invasive tongue base surgeries that will avoid the intraoperative and postoperative complications of the traditional tongue base reduction surgeries

The stapler can be either disposable or reusable, made of plastic or stainless steel. Both types are generally loaded using replaceable disposable cartridges.

The stapler can be either of child or adult size according to the length and diameter of both body and stapling cutting segment

The stapler incorporates a knife or cutter, for complete excision and stapling in a single operation.

The stapler cutter can be a conventional one; working as a cold knife or can be attached to harmonic technology; harmonic scalpel to reduce the tongue tissue bleeding

The staple line is either straight or slightly cutved

The following detailed description should be read with reference to the drawings in which similar elements in different drawings are numbered the same.

Description of the Tongue Debulking Stapler and the Tissue Holding Forceps

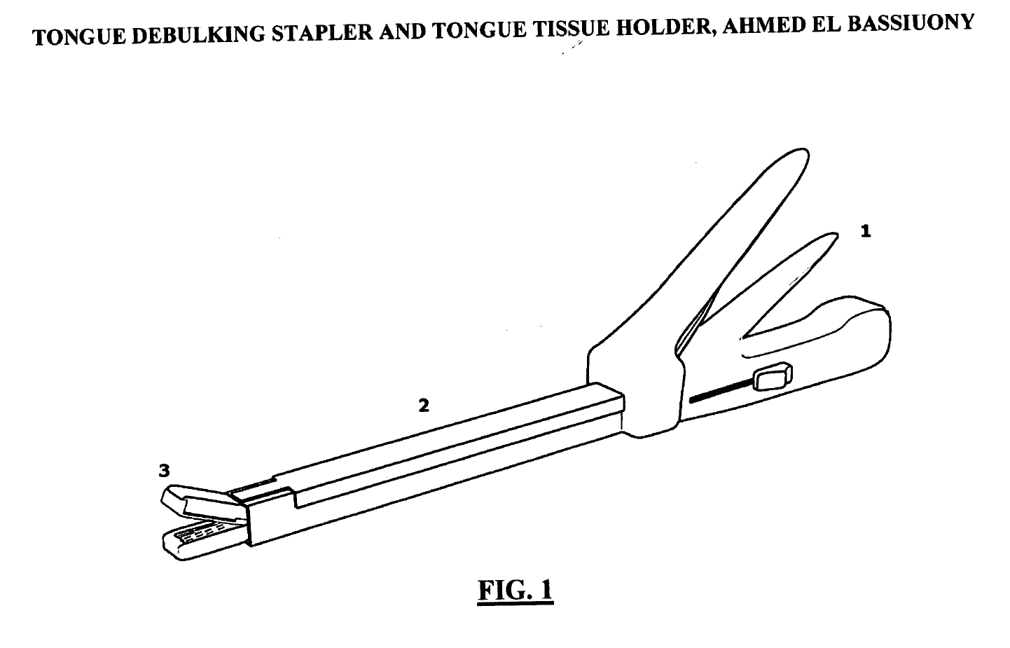

With reference to FIG. 1: the shape and parts of the tongue debulking stapler; side view; vertical orientation.

1 handle
2 shaft
3 stapling cutting part.
Number 1: refers to the handle in all pictures; number 2 refers to the body in all pictures & number 3 refers to the stapling cutting part in all pictures

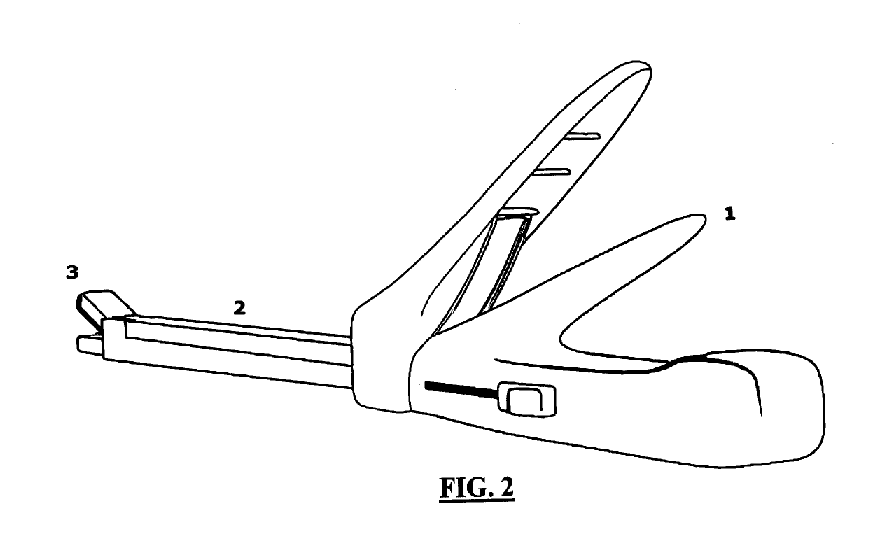

With reference to FIG. 2: showing another side view and parts of the tongue debulking stapler; vertical orientation.

1 handle
2 shaft
3 stapling cutting part

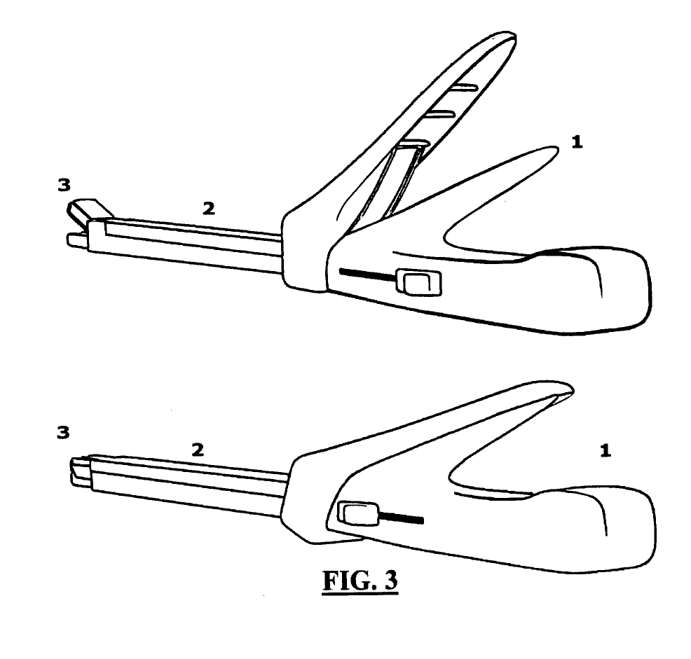

With reference to FIG. 3: schematic diagram showing two views together; one while the handle is opened and the other one while the handle is closed.

1 handle
2 shaft
3 stapling cutting part

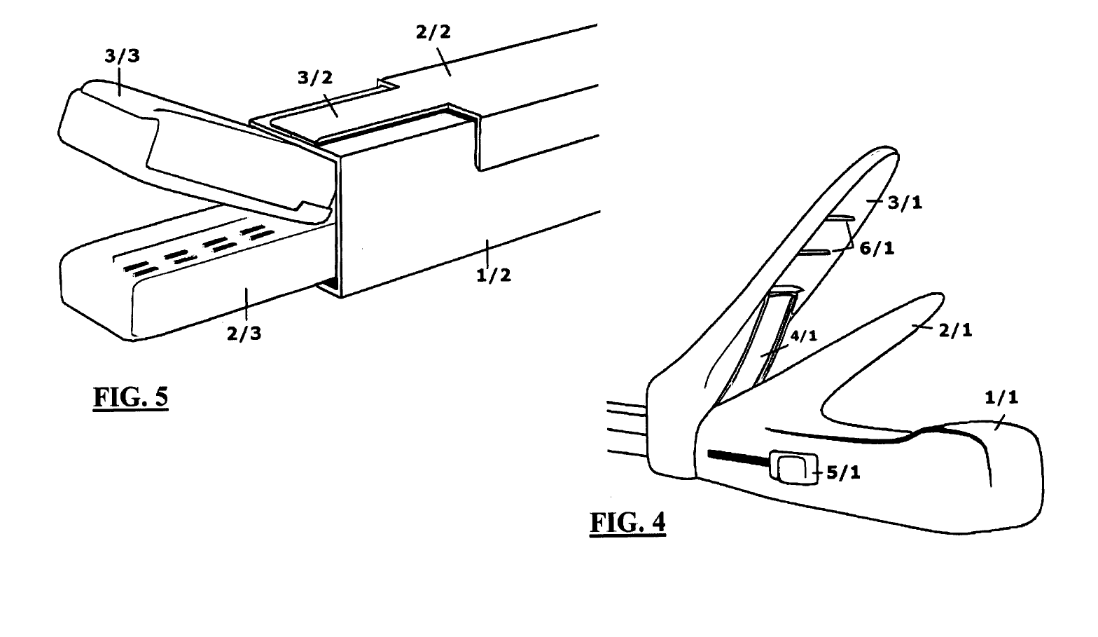

With reference to FIGS. 4 & 5: schematic diagrams showing parts of the handle with view of the stapler cutting segment while the handle opened.

Wwith reference to FIG. 4:
1/1 & 2/1 Fixed grips
3/1 Active grip, working in relation to the fixed grip 2/2 through an articulating joint.
4/1 Control segment: inner surface of the active grip is attached to a control segment with 2 step movement range; for approximation then complete fixation. This segment is continue anteriorly with a sliding bar
5/1 Firing knob: responsible for cutting and stapling as a single function. range of movement is one and half cm in a forward direction. This knob has to 2 step movement mode to insure safe secure cutting
6/1 Two ridges on the inner surface of the active grip needed for the 2 step movement range of the control segment with reference to FIG. 5:
1/2 The stapler shaft
2/2 The sliding bar of stapler body
3/2 The distal end of the sliding bar
2/3 Fixed limb of the stapling segment; anvil
3/3 Mobile limb; cartilage holding limb

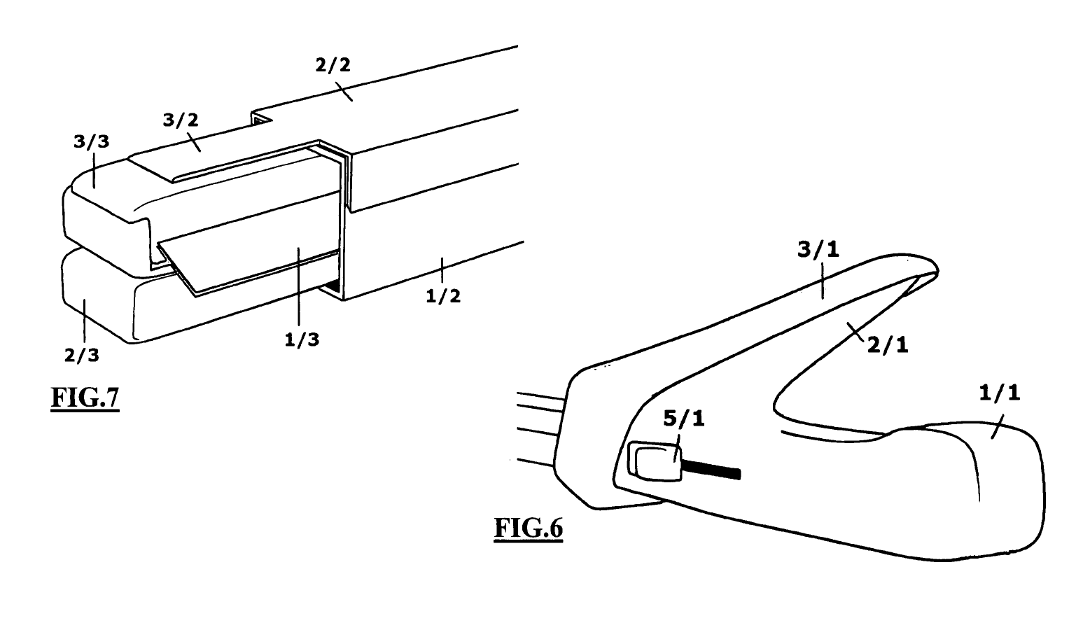

With reference to FIGS. 6 & 7: schematic diagrams showing parts of the handle with view of the stapler cutting segment while the handle closed

With reference to FIG. 6:
1/1 & 2/1 Fixed grips
3/1 Active grip
Movement of the active grip in inwards direction to close with the fixed part through 2 step movement of the control segment
5/1 Firing knob
With reference to FIG. 7:
1/2 The stapler shaft
2/2 The sliding bar of stapler shaft
3/2 The distal end of the sliding bar
2/3 Fixed limb of the stapling segment; anvil
3/3 Mobile limb; cartilage holding limb

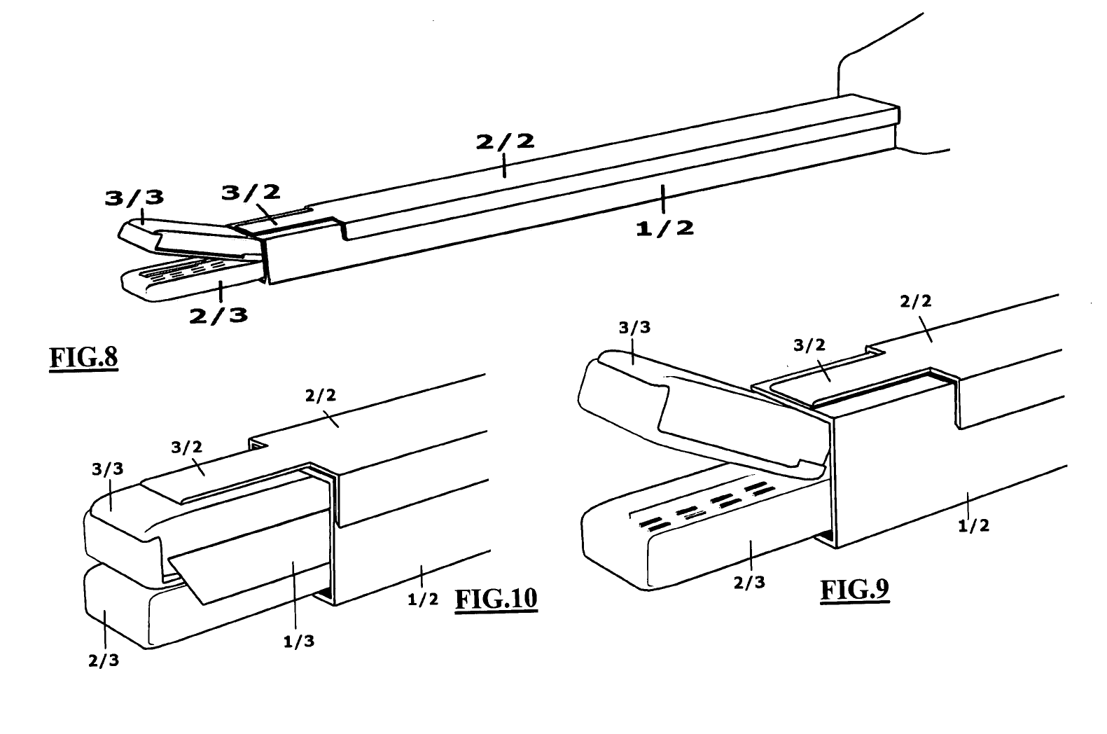

With reference to FIGS. 8, 9 & 10: Schematic diagrams showing parts of the staper body and movement of the sliding bar in relation to the stapling cutting segment; resting position and while moving forewords when the handle closed with reference to FIG. 8: the body and sliding bar:

1/2 The shaft of the stapler
2/2 The sliding bar that work between the control segment of the handle and the cartilage holding limb of the stapling cutting part of the stapler.
3/2 The distal end of the bar is less in wideth than the rest of the bar
2/3 Fixed limb of the stapling segment; anvil
3/3 Mobile limb; cartilage holding limb

Body length: 12-15 cm; external wideth: 5-8 mm and hight: 5 mm; according to the stapler size (child or adult)

With reference to FIG. 9: stapler cutting segment; open:

1/2 The shaft of the stapler
2/2 The sliding bar of stapler shaft
3/2 The distal end of the sliding bar
With reference to FIG. 10: stapler cutting segment; closed
1/2 The body of the stapler
2/2 The sliding bar of stapler shaft
3/2 The distal end of the sliding bar
1/3 Cutting blade
2/3 Fixed limb of the stapling segment; anvil
3/3 Mobile limb; cartilage holding limb

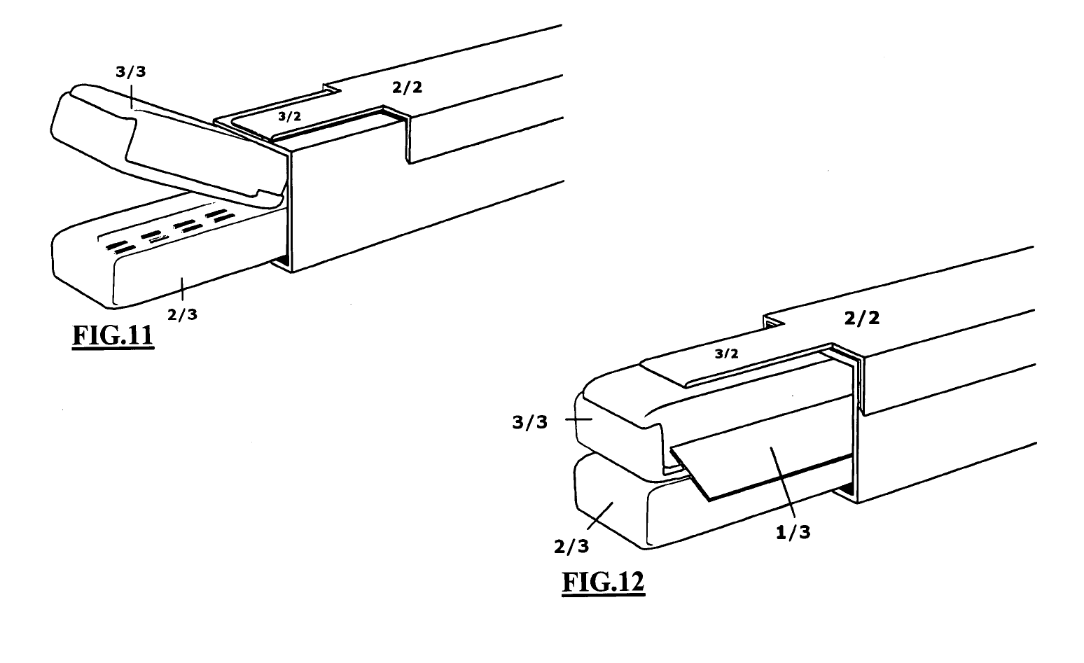

With reference to FIGS. 11 & 12: schematic diagrams showing the stapling cutting segment; opened and closed with reference to FIG. 11: the stapling cutting segment; opened:

2/2 The sliding bar of stapler shaft
3/2 The distal end of the sliding bar
2/3 Fixed limb of the stapling segment; anvil
3/3 Mobile limb; cartilage holding limb. Staples applied in a side-to-side fashion
With reference to FIG. 12: The stapling cutting segment; closed:
2/2 The sliding bar of stapler shaft
3/2 The distal end of the sliding bar
1/3 Cutting blade: running immediately above the stapling part

### Stapling Segmentt:

### Parts:

2/3 Fixed limb of the stapling segment; anvil: width 3 mm
3/3 Mobile limb; cartilage holding limb; to which staples cartilage is attached. Width 6 mm

Each cartilage contains 2 lines of staples; each one consists of 3-4 staples. This part move in relation to the side of distal end of the shaft through a joint

### Length: 15-20 mm

Movement of the movable part of the active grip of the handle leads to a forwards movement of the sliding bar that will push and close the movable part of the stapler working segment. The sliding bar is moving on the external surface of the side of the stapler shaft.

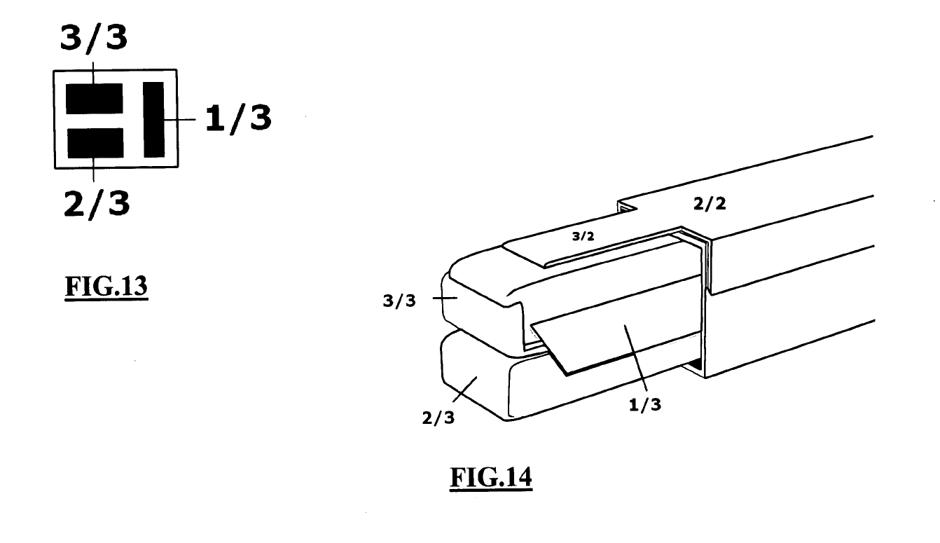

With reference to FIGS. 13 & 14: Schematic diagrams showing front view of the stapling cutting segment; while the stapler is in a vertical position

With reference to FIG. 13:
1/3 Cutting blade
2/3 Fixed limb of the stapling segment; anvil
3/3 Mobile limb; cartilage holding limb
With reference to FIG. 14:
2/2 The sliding bar of stapler shaft
3/2 The distal end of the sliding bar
1/3 Cutting blade
2/3 Fixed limb of the stapling segment; anvil
3/3 Mobile limb; cartilage holding limb

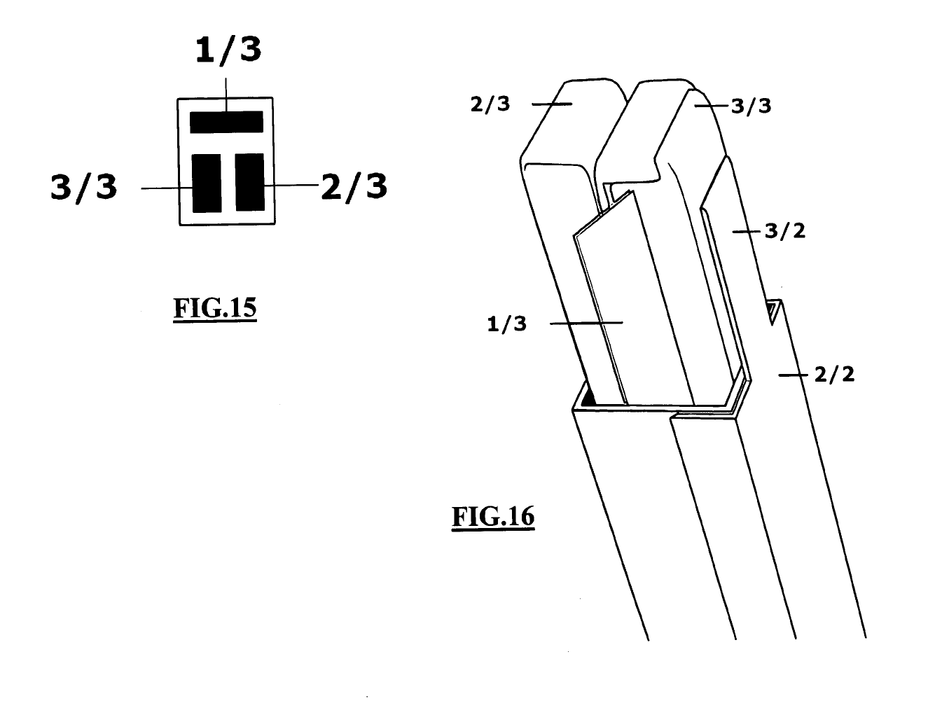

With reference to FIGS. 15 & 16: schematic diagrams showing front view of the stapling cutting segment; while the stapler is in horizontal L position

With reference to FIG. 15:
1/3 Cutting blade
2/3 Fixed limb of the stapling segment; anvil
3/3 Mobile limb; cartilage holding limb
With reference to FIG. 16:
1/2 The sliding bar of stapler shaft
2/2 The distal end of the sliding bar
1/3 Cutting blade
2/3 Fixed limb of the stapling segment; anvil
3/3 Mobile limb; cartilage holding limb

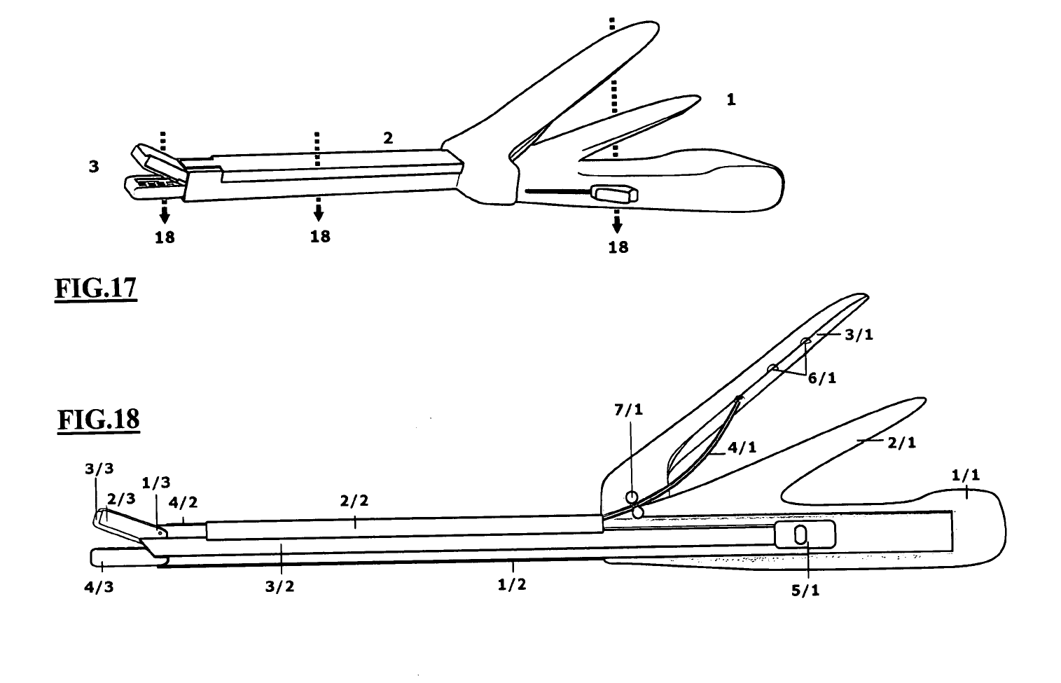

With reference to FIGS. 17 & 18: schematic diagrams showing the direction of the cut section and the cut section of the stapler while the stapler is in vertical position with reference to FIG. 17: the stapler with broken arrows to show the direction of the cut section, vertical orintation of the stapler. 1 handle, 2 shaft and 3 stapling cutting part

With reference to FIG. 18: the cut section
1/1 & 2/1 Fixed grips
3/1 Active grip
4/1 Control segment
5/1 Firing knob
6/1 Two ridges on the inner surface of the active grip
7/1 Acting as contol guide for movement of the contol segment & articulating joint between the 2^{nd} fixed grip and the active grip
1/2 Shaft of stapler

### 2/2 Sliding bar

3/2 Cutting blade
4/2 Distal end of the sliding bar
1/3 Joint for movement of the cartiolage holding limb
2/3 Cartiolage holding limb

### 3/3 Cartilage

### 4/3 Anvil

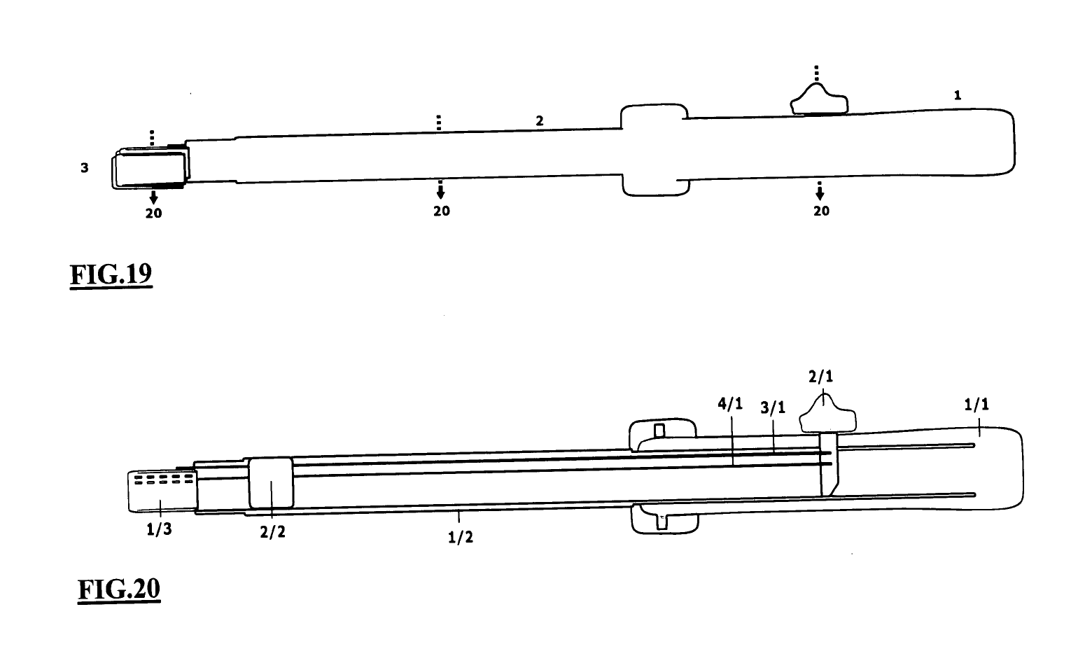

With reference to FIGS. 19 & 20: schematic diagrams showing the direction of the cut section and the cut section of the stapler while the stapler is in horizontal position with reference to FIG. 19: the stapler with broken arrows to show the direction of the cut section, horizontal orintation of the stapler. 1 handle, 2 body and 3 stapling cutting part

With reference to FIG. 20: the cut section
1/1 Fixed grip
2/1 Firing knob
3/1 Cutting blade
4/1 Meatal limb that puch the staples out when entering the cartilage
1/2 Shaft of the stapler
2/2 A plastic strut to which attached the cutter, in its upper anterior end, and the pushinglimb of the staples out when entering the cartilage, in its lower anterior end. The upper surface of the strut is fixed to the inner surface of the stapler body
1/3 Stapling segment

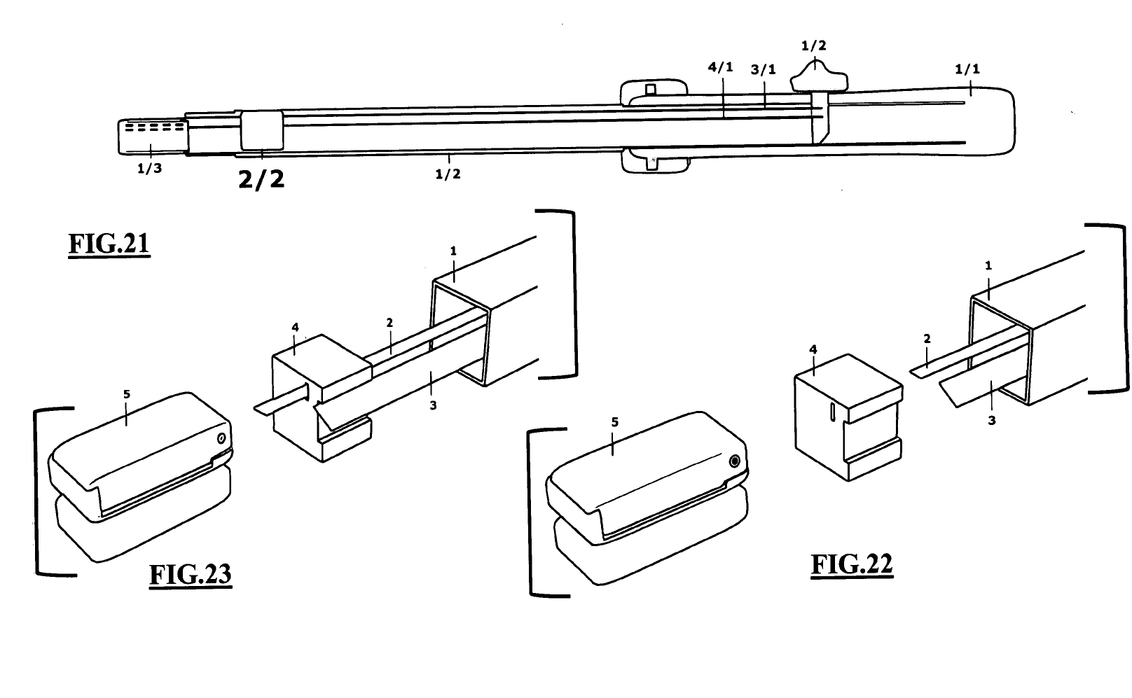

With reference to FIGS. 21, 22 & 23: schematic diagrams showing projection views of the strut holding the cutter and the pushing arm of the staples

With reference to FIG. 21: cut section with projection of strut holding the cutter and the pushing arm of the staples
With reference to FIGS. 22 & 23: projection views of the strut holding the cutter and the pushing arm of the staples
1 Shaft of the stapler
2 Cutting blade
3 The pushing arm of the staples
4 The plastic strut
5 The cartilage holding part of the stapling segment

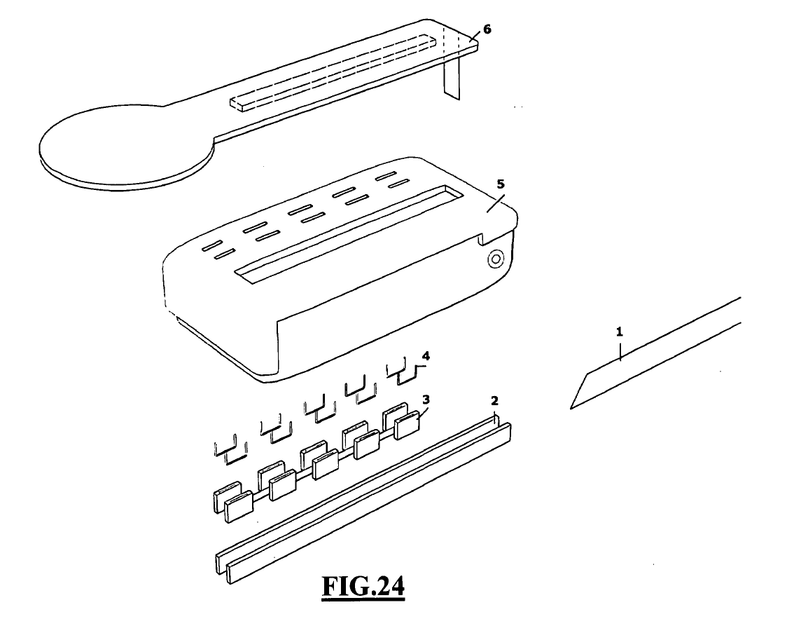

With reference to FIG. 24: a schematic diagram showings the details of the staples holding segment

1 Cutting blade
2 Staples contaner
3 Staples pocket

### 4 Staples

5 The cartilage holding part of the stapling segment
6 The cover protection plastic piece; guide aganst stapler firing

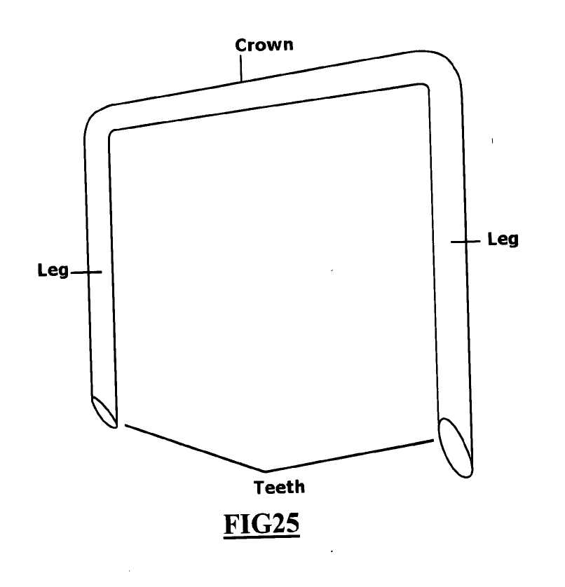

With reference to FIG. 25: a schematic diagram showing of the staples
Made from absorbable material
Diminutions:
   Crown: 4 mm
   Legs: open stapler height 5 mm, closed stapler height 2 mm
   Wire diameter: 1 mm
Release of 3-4 staples at a time

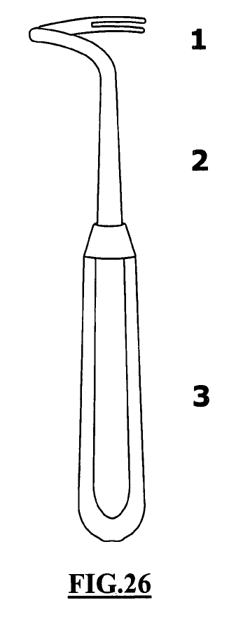

With reference to FIG. 26: a schematic diagram showing of the parts of the tongue tissue holder. This tissue holding will make the stapler work easier and can control the amount of tongue tissue to be excised

1 TIP: Curved in a way to be able to visualise the tongue tissue needed to be excised; with two sharp teeth for holding the tongue tissue. Height in relation to the shaft: 3 mm
2 Shaft: 12-15 cm in length and 4 mm in diameter and more slopping and less in diameter towards the holding tip

### 3 Handle

The following is detailed description of the steps of tongue tissue debulking or partial glossectomy using the tongue debulking stapler

The procedure under taken under general anesthesia

A retraction suture is placed through the anterior midline tongue tip. Mark the boundaries of the lingual artery. A handpiece ultrasound probe can be used to delineate and marks thelingual arteries bilaterally. It is documented in several previous studies that the lingual artery runs laterally along the tongue base and that it is at a relatively consistent distance away from the foramen cecum in an inferior and lateral direction. The lingual artery is consistently medial to both the hypoglossal and lingual nerves

The tongue tissue holder is applied to the area of the tongue needed to be excised. The tongue tissue is pulled upwards and the jaws of the stapler working segment is applied on both sides and closed aganist tongue tissue to be resected. The firing knob is pushed in a forwards direction to cut the tongue tissue and apply staples immediately inferior to the cut line

A layer of surgical glue is applied the cut edge of the tongue to cover the raw area and help in haemostasis

The same is applied to the other side of the tongue. It can be one stage operation or a staged operation, one side of tongue in each session to avoid bilateral tongue oedema which may affect the upper airway especially in infants and children

This technique provides minimal tissue handling with less postoperative tongue edema and short postoperative hospital stay; minimally invasive technique

What is claimed is:

1. A device for tongue size reduction, comprising:
tongue tissue debunking and tissue stapling in the same function

2. A device as in claim 1, introduced for the first time in the field of tongue debunking surgery including osa surgeries as reviewed through midline network with no data available before about this issue

3. A device as in claim 1, which may have 2 sizes; adult one and child one

4. A device as in claim 1, which can be manufactured as disposable or reusable one

5. A device as in claim 1, which can use the harmonic technology as an additional tool attached to the stapler knife in tongue tissue excision

6. A device as in claim 1, which has a special design to be suitable for the area of the oral cavity and it is different from other staplers, used in general surgery

7. A device as in claim 1, the handle has 2 fixed grips with a movable grip working in relation to one of them and attached to a working segment that moves the sliding bar which has 2 movement steps

8. A device as in claim 1, the handle contain a fairing knob which control the movement of both of the knife and the metal limb responsible for fairing of staples

9. A device as in claim 1, the body has a sliding bar which connected to the control segment of the active grip of the stapler handle; closure of which pushes the sliding bar forewords resulting in closure of the movable part of the stapling segment

10. A device as in claim 1, the body has aplastic strut that control and guides the movement of the cutter and the meatal limb responsiple for staples firing and attached to the inner surface of the body

11. A device as in claim 1, the stapling cutting segment which consists of the cutting knife and the stapling segment

12. A device as in claim 1, the stapling segment consists of 2 parts: the fixed anvil and the movable cartilage holding part with a replaceable cartilage containing 2 raw of staplers each one of them consists of 3-4 staplers and can do stapling of 1 mm thickness tissue

13. A device as in claim 1, the stapling segment has a tissue retainer which ensures correct cutting and stapling

14. A device as in claim 1, staples made from absorbable material with special diminutions and released in 3-4 staples at a time

15. A device as in claim 1, supplied by tongue tissue holder to facilitates the stapler work

16. A device and method for tongue size reduction, introducing a new minimally invasive technique in the field of tongue size reduction especially in treatment of OSA

17. A device and method as in claim 16, which can be one stage operation or a staged operation, one side of tongue in each session to avoid bilateral tongue oedema which may affect the upper airway especially in infants and children

18. A device and method as in claim 16, where the tongue tissue is resected and stapled in a single fuction by movement of the firing knob

19. A device and method as in claim 16, control of hemostasis is obtained by the staples itself and application of a layer of a surgical glue on the cut edge of the tongue tissue

20. A device and method as in claim 16, that provides minimal tissue handling with less postoperative tongue edema and short postoperative hospital stay; minimally invasive technique; minimal invasive technique
